(19) United States
(12) Patent Application Publication
(10) Pub. No.: US 2025/0299773 A1
(43) Pub. Date: Sep. 25, 2025

(54) SYSTEMS AND METHODS FOR IDENTIFYING CROSS-SPECIES GENE AND GENE VARIANT RELATIONSHIPS

(71) Applicant: Elissa Chesler

(72) Inventor: Elissa Chesler

(21) Appl. No.: 18/874,860

(22) PCT Filed: Jun. 16, 2023
(86) PCT No.: PCT/US2023/025530

§ 371 Date: Dec. 13, 2024

Related U.S. Application Data
(60) Provisional application No. 63/352,874, filed on Jun. 16, 2022.

(51) Int. Cl. G16B 20/20; G06F 16/901

(52) U.S. Cl. CPC G16B 20/20; G06F 16/9024

(57) ABSTRACT

Systems and methods for generating a graph describing cross-species relationships between gene variants associated with two or more species are provided herein. The techniques include obtaining data generated by first genomic studies of a first species and second genomic studies of a second species, the data including a plurality of datasets including two or more data formats. A subset of the data is stored in a cache and then transformed into a database having a uniform data format, the database describing graph objects and connections between the graph objects. The database is built by iteratively caching and transforming subsets of the data, each transformed subset being stored in non-transient computer-readable memory. The graph is then generated using the database.

### CROSS REFERENCE TO RELATED APPLICATIONS

This application claims the benefit under 35 U.S.C. § 119 (e) of U.S. Provisional Patent Application No. 63/352,874 filed Jun. 16, 2022 and titled “Systems and Methods for Identifying Cross-Species Gene and Gene Variant Relationships,” which is incorporated herein by reference in its entirety.

### STATEMENT REGARDING FEDERALLY-SPONSORED RESEARCH AND DEVELOPMENT

This invention was made with government support under R01 Aa018776, U54 OD020351, DA 037927, and DA 039841, each awarded by the National Institutes of Health. The government has certain rights in the invention.

### BACKGROUND

Model organism research has generated extensive genomic data that can provide insight into the biological mechanisms of gene and gene variant action. Genome-wide association studies and other discovery genetics and genomics methods provide a means to identify previously unknown biological mechanisms underlying diseases, traits, and disorders.

### SUMMARY

Some embodiments provide for a method of generating a graph comprising connections between a first set of gene variants associated with a first species and a second set of gene variants associated with a second species different than the first species, the method comprising: using at least one computer hardware processor to perform: obtaining data generated by first genomic studies of the first species and second genomic studies of the second species, the data comprising a plurality of datasets comprising two or more data formats; storing a subset of the data in a cache; accessing the subset of the data in the cache and transforming the subset of the data into a database having a uniform data format, the database describing graph objects and connections between the graph objects; storing the database in non-transient computer-readable memory; and generating the graph using the database.

Some embodiments provide for at least one non-transitory computer readable storage medium storing processor-executable instructions that, when executed by at least one processor, cause the at least one processor to perform a method of generating a graph comprising connections between a first set of gene variants associated with a first species and a second set of gene variants associated with a second species, the method comprising: obtaining data generated by first genomic studies of the first species and second genomic studies of the second species, the data comprising a plurality of datasets comprising two or more data formats; storing a subset of the data in a cache; accessing the subset of the data in the cache and transforming the subset of the data into a database having a uniform data format, the uniform data format describing graph objects and connections between the graph objects; storing the database in non-transient computer-readable memory; and generating the graph using the database.

Some embodiments provide for a system for generating a graph comprising connections between a first set of gene variants associated with a first species and a second set of gene variants associated with a second species. The system comprises: at least one processor; and at least one non-transitory computer readable storage medium storing processor-executable instructions that, when executed by the at least one processor, cause the at least one processor to perform a method. The method comprises: obtaining data generated by first genomic studies of the first species and second genomic studies of the second species, the data comprising a plurality of datasets comprising two or more data formats; storing a subset of the data in a cache; accessing the subset of the data in the cache and transforming the subset of the data into a database having a uniform data format, the uniform data format describing graph objects and connections between the graph objects; storing the database in non-transient computer-readable memory; and generating the graph using the database.

In some embodiments, determining the graph objects and the connections comprises: determining first gene objects and first gene variant objects associated with the first species; determining second gene objects and second gene variant objects associated with the second species; determining first connections between a gene object of the first gene objects and one or more gene variant objects of the first gene variant objects; determining second connections between a gene object of the second gene objects and one or more gene variant objects of the second gene variant objects; and determining third connections between the first gene objects and the second gene objects.

In some embodiments, determining the first and/or second connections comprises determining expression quantitative trait loci (eQTL), gene variant regulatory elements, chromatin contact regions, and/or intragenic mapping connections.

In some embodiments, determining the third connections comprises determining homolog and/or orthologue connections between the first and second species.

In some embodiments, generating the graph comprises generating a weighted undirected graph.

In some embodiments, the method further comprises: obtaining data generated by third genomic studies of a third species, the third species being different than the first species or the second species; determining third gene objects and third gene variant objects associated with the third species; determining fourth connections between a gene object of the third gene objects and one or more gene variant objects of the third gene variant objects; and determining fifth connections between the third gene objects and the first or second gene objects.

In some embodiments, the method further comprises identifying, using the graph and a gene variant object associated with the first species, one or more genomic studies associated with the second species.

In some embodiments, using the graph to identify the one or more genomic studies comprises: identifying, using the gene variant object associated with the first species, a gene object of the first gene objects using the first connections; identifying a gene object of the second gene objects using the third connections; identifying a gene variant object associated with the second species using the second connections; and identifying the one or more genomic studies using the gene variant associated with the identified gene variant object.

In some embodiments, the gene variant object associated with the first species is associated with a disease, and wherein the method further comprises identifying a treatment modality for a patient having the disease using the identified one or more genomic studies.

In some embodiments, the first species and the second species comprise two species selected from a list including: Mus musculus, Homo sapiens, Rattus norvegicus, Danio rerio, Drosophilia melanogaster, Macaca mulatta, Caenorhabditis elegans, Saccharomyces cervisiae, Gallus gallus, and Canis familiaris.

In some embodiments, the first species and the second species comprise Mus musculus and Homo sapiens.

In some embodiments, transforming the subset of data into a uniform data format comprises transforming the subset of data into one or more comma-separated values (CSV) files.

In some embodiments, obtaining the data comprising two or more data formats comprises obtaining data comprising two or more of a gene transfer file (GTF) format, a genome variation format (GVF), browser extensible data (BED) file format, an EXCEL binary file format (XLS), a comma-separate values (CSV) file format, a tab-separated values (TSV) file format, and/or a report (RPT) file format.

In some embodiments, the method further comprises regenerating the graph based on updated data, the regenerating comprising: obtaining the updated data generated by first genomic studies of the first species and second genomic studies of the second species, the updated data comprising a plurality of datasets comprising two or more data formats; storing a subset of the updated data in a cache; accessing the subset of the updated data in the cache and transforming the subset of the updated data into a uniform data format, the uniform data format describing graph objects and connections between the graph objects; storing, in non-transient computer-readable memory, the transformed subset of the updated data in a database; and regenerating the graph using the database.

In some embodiments, determining the graph objects and the connections comprises: determining first transcript objects associated with the first species; determining sixth connections between the first transcript objects and the first gene objects; and determining seventh connections between the first transcript objects and the first gene variant objects.

In some embodiments, determining the graph objects and the connections comprises: determining first peak objects associated with the first species; and determining eighth connections between the first peak objects and the first gene variant objects.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic diagram of a facility for generating a graph describing gene variant relationships between two or more species, in accordance with some embodiments described herein.

FIG. 2 illustrates a processing pipeline for generating a graph, in accordance with some embodiments described herein.

FIG. 3 is a schematic diagram of input data and output transformed data, in accordance with some embodiments described herein.

FIG. 4A is a schematic diagram of an illustrative graph describing gene variant relationships between two species, in accordance with some embodiments described herein.

FIG. 4B is a schematic diagram of an illustrative graph describing gene variant relationships between three species, in accordance with some embodiments described herein.

FIG. 4C is a representation of a branch of a graph for one species in Cypher graph notation, in accordance with some embodiments described herein.

FIG. 5 is a schematic diagram of illustrative data sources and an illustrative graph describing gene variant relationships between mice and humans, in accordance with some embodiments described herein.

FIG. 6 is a flowchart of an illustrative process 600 for generating a graph, in accordance with some embodiments described herein.

FIG. 7 is a schematic diagram of an integrated software system including a graph describing gene variant relationships between two or more species, in accordance with some embodiments described herein.

FIG. 8 is a schematic diagram of an illustrative computing device with which aspects described herein may be implemented.

### DETAILED DESCRIPTION

Described herein are techniques for generating a graph structure describing gene variant relationships across two or more species. Such a graph structure may be used to identify biological studies performed in one species that may be relevant to a condition, disease, or disorder in another species, thereby enabling potential identification of new treatment modalities of the condition, disease, or disorder. Because genomic data for a species can occupy terabytes of storage, these techniques include methods of streaming portions of the genomic data and storing the data portions in a cache to efficiently utilize computing resources. Additionally, the techniques described herein to stream portions of the genomic data into a temporary cache have been found to accelerate the process of generating the graph. For example, a two-species graph including approximately 3.6 billion relationships may be generated in approximately 18 hours. In contrast, without using the techniques described herein, a two-species graph including approximately 1.7 million relationships required approximately one week to be generated. The improved graph generation speed allows for the frequent and fast updating of the graph in response to the publication of new genomic studies or updates to major sources of data, such as the release of revised versions of Ensembl.

In some embodiments described herein, the cached data portions may initially be stored in diverse, sometimes incompatible, data formats. These diverse data formats may be transformed into a uniform data format before building the graph. The uniform data format may include the graph objects (e.g., genes, gene variants, and other objects) and the connections between the graph objects which form the gene variant relationships of the graph. The graph may then be built using the data stored in the uniform data format. In this manner, the graph may be quickly and efficiently built and updated.

Genome-wide association studies (GWAS) and other discovery genetics methods provide a means to identify previously-unknown biological mechanisms underlying diseases, disorders, and other health conditions. GWAS may point to new therapeutic avenues and/or diagnostic tools and may yield a deeper understanding of the biology of a variety of health conditions. However, the predictive power of GWAS and other discovery genetics can be dependent on the sample size of the genomic study. Power analyses show that the massive polygenicity underlying relevant traits and illnesses requires larger sample sizes for additional discoveries when relying on GWAS data alonc. Likewise, the predictive power of a polygenic risk score (PRS), an index of aggregated genetic susceptibility to a disorder, for certain disorders is also directly linked to the current statistical power of discovery GWAS.

The inventors have recognized and appreciated that genetic studies of model organisms may improve the predictive power of GWAS by augmenting the genomic study sample size and may therefore provide insight into human traits and/or diseases. However, there remain conceptual and technical challenges for data integration across species. While cross-species analysis typically happens at the level of abstracted relations among variants or genes and can be reduced in scale, the scope of genomic studies is comparatively unbounded and it can be possible to find hundreds, if not thousands of animal studies of disease-relevant biology. The computational parsing and representation of genomic variants from diverse data sources and their mappings onto one another does not easily scale and retaining a traceable mapping while allowing integrative and interactive analysis is a problem of high complexity. Further, the storage, analysis, distribution, and integration of human and model organism functional genomic data are especially challenging, as they embody typical problems encountered in the big data world referred to as the four V's of data: volume, variety, velocity, and veracity.

First, the sheer volume and variety of data required to support comprehensive cross-species data integration of genes and individual variants is staggering. For example, if the average number of coding genes in mammalian genomes is assumed to be approximately 25,000, then constructing rudimentary connections among the genes in five species would produce 1/2n(n−1) relationships, where n is the number of genes in the network. If represented as a graph, with each edge representing a relationship, the graph would be enormous but tractable, comprising approximately 7.8×10⁹ edges. But the genome is only one dimension of the problem; the other is the sheer number of contexts in which that genome is experimentally profiled. With thousands of human and model organism genomics datasets, and hundreds of thousands of species-specific pathway data, organ regional transcriptomes and other relevant data resources, one quickly reaches a problem requiring scalable solutions. Additionally, at the gene variant level, the relationship problem is greatly compounded. Known variants, which outnumber genes within the typical model organisms by more than 20,000-to-1, would naively include approximately 1.25×10¹⁷ edge relationships.

While intelligent approaches for computing on large graphs, such as taking advantage of partitioning, sparse connectivity, or heuristics, can aid in the management and analysis of these relationships, exhaustive examination of static graphs of this potential size is intractable due to computing limitations, storage, and real-time accessibility. As the number of genomic experiments continues to grow, particularly in the model organism space, the inventors have recognized that dynamic analysis of datasets may be performed using horizontally-scalable computing, which can efficiently distribute computing tasks to address very specific genomic questions.

Second, in addition to the large volume and variety of data associated with gene variant mapping across species is the velocity at which the data is produced, and, subsequently, the rate at which the data must be collated, curated, and made accessible. With over 4500 eukaryotic genomes assembled over the last decade, it has been argued that genome-scale data will be bigger than Big Data associated with astronomy, YouTube, and Twitter by 2025. Furthermore, the processes used to integrate the vast scope of data are data sharing policies that historically do not require automated sharing of model organism data, resulting in data analysis processes that result primarily from ad hoc relationships. To mitigate the stresses imposed by data velocity, the inventors have recognized that accessing, integrating, and dynamically updating these data should be performed in a manner that avoids redundancies and keeps data provenance intact.

The inventors have accordingly developed computationally-efficient systems and methods for generating graphs describing gene variant relationships across species. The techniques described herein are configured to parse publicly available genomic data resources using data streaming. This streamed data may then be collated into a specific data format for bulk import into a graph database. Intermediate relational databases may be configured to store data during this process, as the scale of the data may be too large to fit in computer memory. The resulting graph database may have on the order of tens of billions of nodes and relationships.

In some embodiments, the graph includes connections describing relationships between a first set of gene variants associated with a first species and a second set of gene variants associated with a second species different than the first species. For example, in some embodiments, the first species and the second species may be selected from one of the following species: Mus musculus, Homo sapiens, Rattus norvegicus, Danio rerio, Drosophilia melanogaster, Macaca mulatta, Caenorhabditis elegans, Saccharomyces cervisiae, Gallus gallus, and Canis familiaris. In some embodiments, the first species may be Mus musculus and the second species may be Homo sapiens. In some embodiments, the first species may be Mus musculus and the second species may be Rattus norvegicus. In some embodiments, the first species may be Canis familiaris and the second species may be Homo sapiens. In some embodiments, the first species and the second species may be selected from the species included in current and/or previous releases of Ensembl (https://www.ensembl.org/).

In some embodiments, the graph is generated using at least one computer hardware processor to obtain data generated by: (1) genomic studies related to the first species and (2) genomic studies related to the second species. The data may be obtained from one or more of locally-stored data (e.g., data stored on local computer memory) and/or remotely-stored data (e.g., data stored on remote computer memory and accessed over a network or the internet). The obtained data may include data that is stored in two or more different data formats. For example, the data may be stored in any combination of a gene transfer file (GTF) format, a genome variation format (GVF), browser extensible data (BED) file format, an EXCEL binary file format (XLS), a comma-separate values (CSV) file format, a tab-separated values (TSV) file format, and/or a report (RPT) file format.

In some embodiments, a subset of the obtained data is “streamed” and stored in a cache (e.g., in a temporary database stored on non-transient computer-readable memory and/or in random-access memory) for processing. The subset of the data is then accessed from the cache and transformed into a uniform data format that is then stored in a database in non-transient computer-readable memory. For example, the subset of the data may be transformed into a database stored in one or more comma-separated values (CSV) files.

In some embodiments, when transforming the subset of the data, descriptions of graph objects (e.g., genes, gene variants, transcripts, etc.) and connections between graph objects (e.g., connections between genes and gene variants or other relationships) may be generated and stored in the database. The graph may then be generated using the graph objects and connections stored in the database. In some embodiments, the graph may be generated as a weighted undirected graph.

In some embodiments, determining the graph objects includes determining gene objects (e.g., genes) and gene variant objects (e.g., gene variants) associated with each of the first and second species. Determining the connections may then include determining connections between the gene objects and gene variant objects within each of the first and second species. In some embodiments, determining the connections between the gene objects and gene variant objects within a species may include determining expression quantitative trait loci (eQTL), gene variant regulatory elements, chromatin contact regions, and/or intragenic mapping connections.

In some embodiments, determining the connections may also include determining cross-species connections between the gene objects of the first and second species. For example, determining the gene object connections between the first and second species may be performed by determining homolog and/or orthologue connections between genes of the first and second species.

In some embodiments, the graph may include connections between three or more species. Generating the graph may additionally include obtaining data generated by third genomic studies of a third species different than the first species or the second species. Determining the graph objects may additionally include determining gene objects and gene variant objects associated with the third species. Determining the connections may additionally include determining connections between gene objects and gene variant objects associated with the third species. Furthermore, determining the connections may additionally include determining connections between gene objects associated with the third species and gene objects associated with the first and/or second species.

In some embodiments, the techniques may include regenerating the graph based on updated data (e.g., due to new or additional genomic studies being performed on the first and/or second species). Regenerating the graph may include obtaining the updated data and storing a subset of the updated data in a cache. The subset of the updated data stored in the cache may then be accessed and transformed into a database stored in one or more files having a uniform data format. The database may be stored in non-transient computer-readable memory and may include information describing graph objects and connections between the graph objects. The graph may then be regenerated using the database.

In some embodiments, the techniques further include identifying, using the generated graph and a gene variant object associated with the first species, genomic studies associated with the second species. For example, a user may provide a gene variant associated with the first species and use the graph to identify a related gene using the connections between gene variant objects and gene objects associated with the first species. The user may then identify a gene object associated with the second species using the cross-species connections between gene objects associated with the first and second species. The user may then identify a gene variant object associated with the second species using the connections between gene variant objects and gene objects associated with the second species. After identifying the related gene variant object associated with the second species, the user may identify genomic studies associated with the identified gene variant object.

In some embodiments, the gene variant object associated with the first species may be associated with a disease, condition, disorder, and/or trait of a patient. The method of using the graph may additionally include identifying a treatment modality for the patient using the identified genomic studies associated with the second species. In this manner, a user may traverse the graph to identify cross-species relationships and genomic studies that may elucidate conditions, diseases, and/or treatments for the first species.

Following below are more detailed descriptions of various concepts related to, and embodiments of, the generation of graphs describing cross-species gene and gene variant relationships. It should be appreciated that various aspects described herein may be implemented in any of numerous ways. Examples of specific implementations are provided herein for illustrative purposes only. In addition, the various aspects described in the embodiments below may be used alone or in any combination and are not limited to the combinations explicitly described herein.

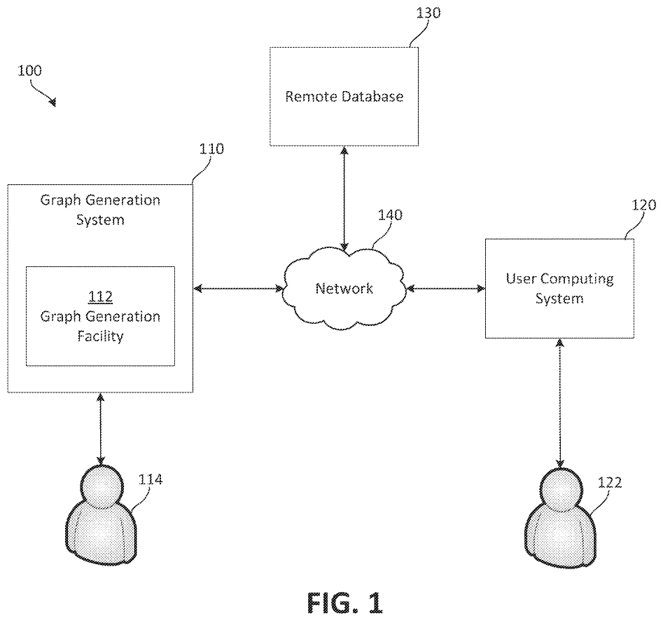

FIG. 1 is a block diagram of an example of a system 100 for generating and using a graph describing relationships between gene variants associated with two or more species, in accordance with some embodiments described herein. In the illustrative example of FIG. 1, the system 100 includes a graph generation system 110, a user computing system 120, and a remote database 130. It should be appreciated that the system 100 is illustrative and that a system may have one or more other components of any suitable type in addition to or instead of the components illustrated in FIG. 1. For example, there may be additional remote databases or additional user computing systems (e.g., two or more) present within a graph generation system.

As illustrated in FIG. 1, in some embodiments, one or more of the graph generation system 110, the user computing system 120, and the remote database 130 may be communicatively connected by a network 140. The network 140 may be or include one or more local- and/or wide-area, wired and/or wireless networks, including a local-area or wide-area enterprise network and/or the Internet. Accordingly, the network 140 may be, for example, a hard-wired network (e.g., a local area network within a facility), a wireless network (e.g., connected over Wi-Fi and/or cellular networks), a cloud-based computing network, or any combination thereof. For example, in some embodiments, the graph generation system 110 and the user computing system 120 may be located within a same facility and connected directly to each other or connected to each other via the network 140, while the remote database 130 may be located in a remote facility and connected to the graph generation system 110 through the network 140. As another example, in some embodiments, the graph generation system 110 and the user computing system 120 may be located in separate, remote facilities and may be connected to one another through the network 140.

In some embodiments, the graph generation system 110 may be configured to generate graphs describing cross-species relationships between genes and gene variants. The graph generation system 110 may be any suitable electronic device configured to receive instructions and/or information from user computing system 120 and/or remote database 130. In some embodiments, the graph generation system 110 may be a fixed electronic device such as a desktop computer, a rack-mounted computer, or any other suitable fixed electronic device. Alternatively, the graph generation system 110 may be a portable device such as a laptop computer, a tablet computer, or any other portable device that may be configured to receive instructions and/or information from user computing system 120 and/or remote database 130 and to process obtained information from user computing system 120 and/or remote database 130.

Some embodiments may include a graph generation facility 112. The graph generation facility 112 may be configured to process genomic study data obtained from local storage and/or from remote database 130. The graph generation facility 112 may be configured to, for example, access and obtain genomic study data having diverse data formats, to transform the genomic study data into a database stored in one or more files having a uniform data format, and to generate one or more graphs based on the database. The graph generation facility 112 may be implemented as hardware, software, or any suitable combination of hardware and software, as aspects of the disclosure provided herein are not limited in this respect. As illustrated in FIG. 1, the graph generation facility 112 may be implemented in graph generation system 110, such as by being implemented in software (e.g., executable instructions) executed by one or more processors of the graph generation system 110. However, in other embodiments, the graph generation facility 112 may be additionally or alternatively implemented at one or more other elements of the system 100 of FIG. 1. For example, the graph generation facility 112 may be implemented at the graph generation system 110. In other embodiments, the graph generation facility 112 may be implemented at or with another device, such as a device located remote from the system 100 and receiving data via the network 140.

The graph generation system 110 may be accessed by an operator 114 in order to initiate a graph generation or regeneration process using graph generation system 110 The operator 114 may implement a graph generation or regeneration process by inputting one or more instructions into the graph generation system 110 (e.g., the operator 114 may select from which locally-stored and/or remotely-stored databases the graph generation system 110 should obtain data to use to build the graph).

As illustrated in FIG. 1, the system 100 includes a user computing system 120 communicatively coupled to the graph generation system 110. The user computing system 120 may be any suitable electronic device configured to send instructions and/or information to the graph generation system 110 and/or to receive information from the graph generation system 110. In some embodiments, the user computing system 120 may be a fixed electronic device such as a desktop computer, a rack-mounted computer, or any other suitable fixed electronic device. Alternatively, the user computing system 120 may be a portable device such as a laptop computer, a smart phone, a tablet computer, or any other portable device that may be configured to send instructions and/or information to the graph generation system 110 and/or to receive information from the graph generation system 110.

The user computing system 120 may be accessed by a user 122 in order to use a graph generated by graph generation system 110. For example, user 122 may implement a query process to identify related gene variants cross-species by inputting one or more instructions into user computing system 120 (e.g., user 124 may provide one or more genes and/or genes related to a species to query the graph).

Examples of some non-limiting user queries are provided below:

### EXAMPLE 1: FINDING LINKS BETWEEN SPECIES' GENES

MATCH (h:Gene {species: “Homo sapiens”}) -- (m:Gene
{species:“Mus musculus”}) RETURN
h.geneName,h.geneId,m.geneId,m.geneName;

This call results in finding all the links between H. sapiens and M. musculus and returns linked genes and their names.

### EXAMPLE 2: GENERATING GENES OF ONE SPECIES AND THEIR BASE PAIR LOCATIONS

MATCH (g:Gene {species: “Homo sapiens”}) RETURN
g.geneId,g.geneName,g.chr,g.start,g.end LIMIT 25;

This call returns all H. sapiens genes and their base pair locations.

### EXAMPLE 3: LINKING GENES TO VARIANTS OF ANOTHER SPECIES WITHOUT AN ORTHOLOGUE

LOAD CSV WITH HEADERS FROM
“https://bitbucket.org/../variants.csv” as row MATCH
(n:Variant ({variantId:
row.id})-[e:VARIANT_EFFECT]-(t:Transcript)-[ ]-(g1:Gene)
WHERE
NOT (g1)-[:ORTHOLOG]-(:Gene) RETURN
n.VariantId,e.sequenceVariant,t.transcriptId,g1.geneId;

This call returns tables of genes linked to the variants in this file where the genes found do not have an orthologue to another gene.

### EXAMPLE 4: DETERMINING EPIGENETIC PEAKS OVERLAPPING A VARIANT LOCATION

MATCH (v:Variant{rsId: “${rs}”})-[o:Overlap]-(p:Peak) RETURN
v.rsId, p.peakId, p.epigenome, p.featureType;

This call returns a table of peaks and associated epigenetic information for a list of variants whose rslds are inserted using $ {rs}.

### EXAMPLE 5: FINDING VARIANTS LINKING ACROSS SPECIES BY TISSUE ASSOCIATED WITH THE BRAIN

MATCH (vfrom:Variant
{rsId:“${rs}”})-[efrom:EQTL]-(gfrom:Gene)-[link:ORTHOLOG
{source: “BAYLOR”}]-(gto:Gene{species:“Homo
sapiens”})-[eto:EQTL]-(vto:Variant) WHERE
(toLower(efrom.tissueName) CONTAINS “brain” OR
toLower(efrom.tissueFileName) CONTAINS “brain” OR
toLower(efrom.tissueGroup) CONTAINS “brain” AND
(toLower(eto.tissueName) CONTAINS “brain” OR
toLower(eto.tissueFileName) CONTAINS “brain” OR
toLower(eto.tissueGroup) CONTAINS “brain”) RETURN
vfrom.rsId,
efrom.tissueName, efrom.tissueFileName, efrom.tissueGroup,
efrom.uberon, efrom.source,
gfrom.geneName,gfrom.geneId,gfrom.species,gto.geneName,gto.
ge
neId,gto.species,eto.tissueName, eto.tissueFileName,
eto.tissueGroup, eto.uberon, eto.source, vto.rsId;

This call traverses the graph from a variant list inserted using $ {rs} in one species to the associated variants in another species. The data is returned as a CSV table with information about each entity used in the traverse, and all eQTL links are limited to those associated with the brain.

Returning to FIG. 1, the graph generation system 110 also interacts with remote database 130 through the network 140, in some embodiments. The remote database 130 may be any suitable electronic device configured to store information and to transmit information to the graph generation system 110. The remote database 130 may be remote from the graph generation system 110 and user computing system 120, such as by being located in a different room, wing, or building of a facility than the graph generation system 110, or being geographically remote from the graph generation system 110 and user computing system 120, such as being located in another part of a city, another city, another state or country, etc. In some embodiments, remote database 130 may be a fixed electronic device such as a desktop computer, a rack-mounted computer, a fixed server, or any other suitable fixed electronic device. Alternatively, remote database 130 may be a portable device such as a laptop computer, a smart phone, a tablet computer, or any other portable device that may be configured to store and transmit information to the graph generation system 110.

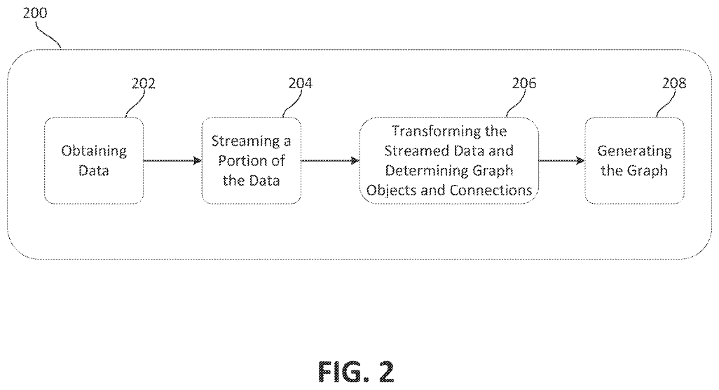

FIG. 2 is a flowchart of an illustrative pipeline 200 for generating a graph describing cross-species relationships between genes and/or gene variants, in accordance with some embodiments described herein. Pipeline 200 may be implemented by a graph generation facility, such as the graph generation facility 112 of FIG. 1. As such, in some embodiments, the pipeline 200 may be used by a computing device configured to access and obtain genomic study data (e.g., graph generation system 110 accessing and obtaining data from remote database 130) and/or to receive queries from remote user devices (e.g., graph generation system 110 receiving queries from user computing system 120). As another example, in some embodiments, the pipeline 200 may be used by one or more processors located remotely (e.g., as part of a cloud computing environment, as connected through a network) from the computing device.

In some embodiments, genomic study data 202 may be obtained at the beginning of the pipeline 200. The genomic study data 202 may be obtained by a computing device from remote and/or local data storage. For example, some portions of the genomic study data 202 may be stored locally on the computer memory of the computing device and some portions of the genomic study data 202 may be stored remotely (e.g., in a computer memory accessible over a network, in cloud computing storage, or otherwise available over the Internet).

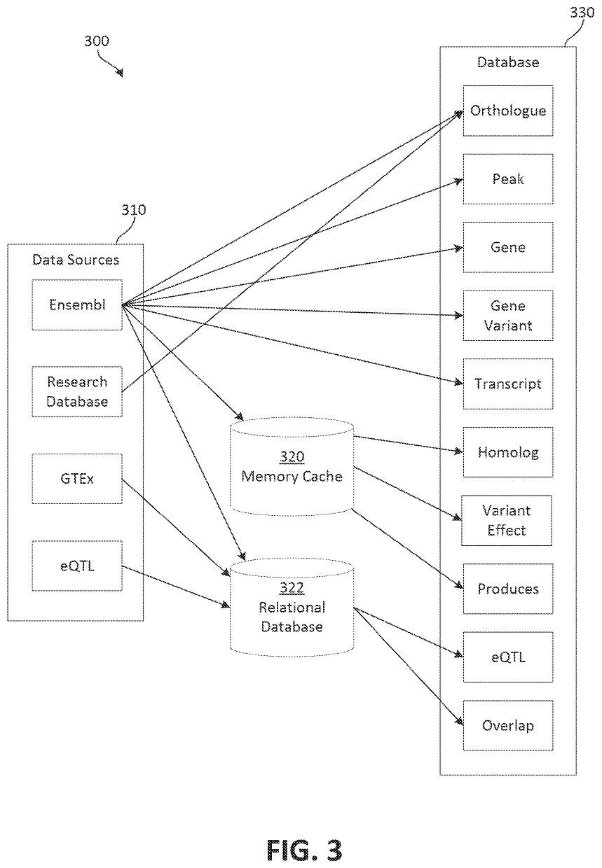

In some embodiments, the genomic study data 202 may be obtained by the computing device from one or more data sources. As illustrated in FIG. 3, the genomic study data 202 may be obtained by the computing device from data sources 310 provided by one or more institutions and/or organizations, including but not limited to Ensembl, the Genotype-Tissue Expression (GTEx) Project, The Jackson Laboratory, the ENCODE Consortium, and/or any other suitable source of genomic study data such as public and private databases.

In some embodiments, the obtained genomic study data 202 may be stored in files having multiple (e.g., two or more) data formats. These multiple data formats may be incompatible with each other and/or difficult to parse. For example, the genomic study data 202 may be stored in files stored using two or more of the following, non-limiting data formats: a gene transfer file (GTF) format, a genome variation format (GVF), a browser extensible data (BED) file format, an EXCEL binary file format (XLS), a comma-separate values (CSV) file format, a tab-separated values (TSV) file format, and/or a report (RPT) file format. It should be appreciated that the genomic study data 202 may be stored in files using alternative data formats than listed herein, as aspects of the technology described herein are not limited in this respect.

In some embodiments, the genomic study data 202 may include genomic study data related to two or more species of organisms. In some embodiments, the species may be selected from a list including the following species: Mus musculus, Homo sapiens, Rattus norvegicus, Danio rerio, Drosophilia melanogaster, Macaca mulatta, Caenorhabditis elegans, Saccharomyces cervisiae, Gallus gallus, and Canis familiaris. For example, in an embodiment where the genomic study data relates to two species, the species may include Mus musculus and Homo sapiens. In some embodiments, the first species may be Mus musculus and the second species may be Rattus norvegicus. In some embodiments, the first species may be Canis familiaris and the second species may be Homo sapiens. In some embodiments, the species may be selected from the species included in current and/or previous releases of Ensembl (https://www.ensembl.org/).

In some embodiments, after obtaining the genomic study data 202, a subset 204 of the genomic study data may be streamed onto the computing device being used to generate the graph. When streamed onto the computing device, the subset 204 may be stored as a cache (e.g., a temporary, easily accessible database). The cache may be stored on any suitable combination of local non-transitory computer-readable memory (e.g., the local hard drive) and/or on local random-access memory (RAM). In some embodiments, storing the cache primarily or wholly on local computer-readable memory may enable generation of the graph using conventional computing resources (e.g., rather than requiring supercomputing resources).

Once streamed onto local memory and/or RAM, the subset of the genomic study data may be transformed into a database 206 stored in one or more files. For example, the database 206 may be stored in local non-transitory computer-readable memory (e.g., the local hard drive) and/or remote non-transitory computer-readable memory (e.g., on the cloud). In some embodiments, the database 206 may be stored in one or more files having a uniform data format. For example, the database 206 may be stored in one or more comma-separated value (CSV) files.

In some embodiments, transforming the subset 204 may include determining graph objects and connections between graph objects to be included in the database 206. As illustrated in FIG. 3, the obtained data from the data sources 310 may be provided to one or both of a memory cache 320 and/or an intermediate relational database 322 to transform the obtained data into the relational database 330. By streaming the data into either the memory cache 320 and/or the intermediate relational database 322, the amount of data in computer memory may be limited such that the input file(s) is never completely stored in computer memory, but rather being read incrementally to preserve computer memory resources.

In some embodiments, determining the graph objects may include determining a number of objects related to genes and gene variants. As illustrated in FIG. 3, the relational database 330 may include gene objects, gene variant objects, transcript objects, and peak objects. These graph objects may be data fields in which additional information describing the gene, gene variant, transcript, and/or genetic peak (e.g., identified using peak calling techniques) is provided. For example, a gene variant object may include data fields describing known genomic studies and/or treatment modalities related to the gene variant label provided for the gene variant object.

In some embodiments, determining the connections may include determining connections that describe relationships between different graph objects (e.g., gene-gene variant relationships, gene-gene relationships, gene-transcript relationships, gene variant-peak relationships, etc.). As illustrated in FIG. 3 and as one example, the relational database 330 may include gene-gene variant relationships defined by “produces” connections that describe a relationship between transcript and gene objects, variant effect connections, and expression quantitative trait loci (eQTL) connections. Additional gene-gene variant relationships, which are not shown in FIG. 3, may include gene variant regulatory elements, chromatin contact regions, and/or intragenic mapping connections. Also as illustrated in FIG. 3, the database 330 may include gene-gene relationships such as, for example, homolog or orthologue connections that are connections made between genes of the different species.

In some embodiments, and returning to the example of FIG. 2, the acts of streaming in subsets 204 of the genomic study data and transforming the subsets 204 may be performed in an iterative process to build the database 206. For example, after transforming a first subset of the data, the cached database storing the first subset may be deleted. Then, a second subset of the data may be streamed into a second cached database so that the second subset of the data may be transformed. The transformed second subset of data may be appended to the database 206. In this manner, the database 206 may be built by streaming and transforming a number of subsets of the genomic study data 202.

In some embodiments, once the genomic study data 202 has been transformed into the database 206, the graph 208 can be generated using the database 206. The graph 208 may be generated as a weighted undirected graph. In some embodiments, the graph 208 may be generated using a Neo4j graph database.

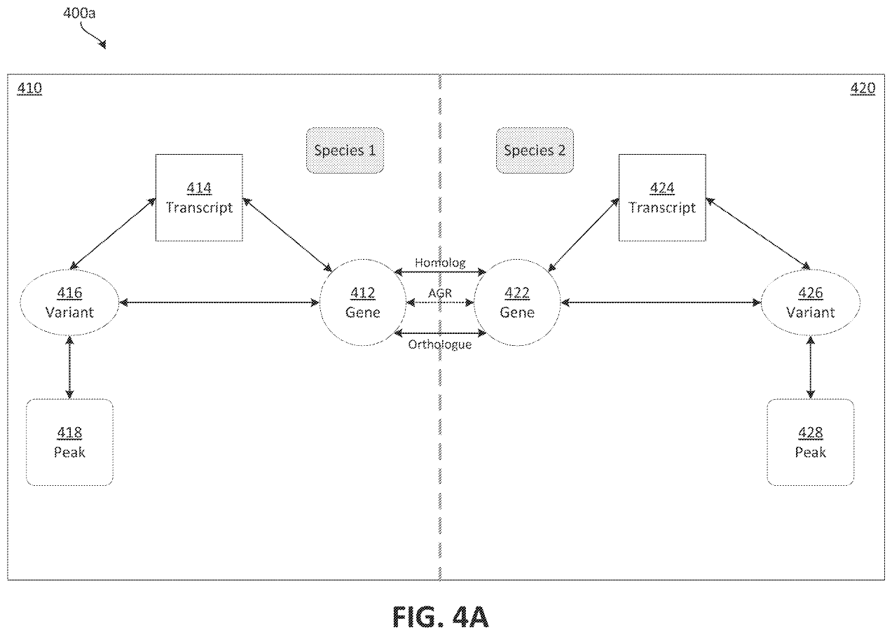

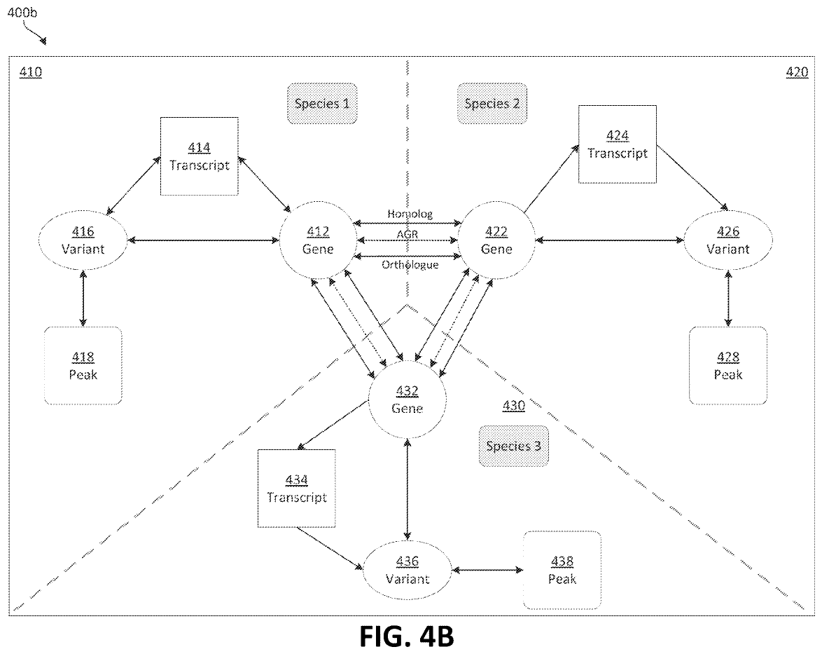

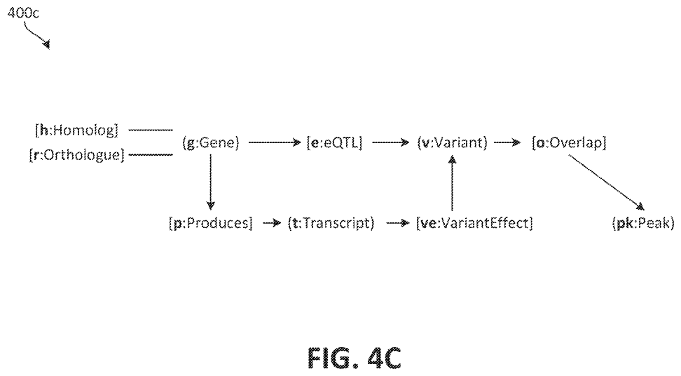

Examples of graph structures are illustrated in FIGS. 4A and 4B. FIG. 4A shows an example of a graph structure for a graph describing relationships between two species (“Species 1” and “Species 2”). FIG. 4B shows an example of a graph structure for a graph describing relationships between three species (“Species 1,” “Species 2,” and “Species 3”). It should be appreciated that the graph 208 is not limited to graphs describing relationships between only two or three species, and that the graph 208 may generated to describe relationships between four or more species, as aspects of the technology described herein are not limited in this respect.

As shown in the example of FIG. 4A and in some embodiments, the graph 400a may include a first portion 410 describing genome relationships within a first species and a second portion 420 describing genome relationships within a second species. The first portion 410 and the second portion 420 may be connected by relationships between the genes 412, 422 of each portion 410, 420. For example, the connecting relationships may include homolog, orthologue, and/or other relationships defined by data from the Alliance of Genome Resources (AGR).

Within each of the first portion 410 and the second portion 420, the genes 412, 422 may be nodes within the graph 400a that are internally connected by edges to transcripts 414, 424 and/or gene variants 416, 426. Additionally, gene variants 416, 426 may be internally linked to transcripts 414, 424 and peaks 418, 428 by additional edges within the graph 400a.

As shown in the example of FIG. 4B and in some embodiments, the graph 400b may be an extension of graph 400a to three species represented by first portion 410, second portion 420, and third portion 430. The genes 432 of the third portion 430 may be linked to the genes 412 and 422 of the first and second portions 410, 420 by orthologue, homolog, and AGR relationships. Within third portion 430, the genes 432 may be connected by edges to transcripts 434 and gene variants 436, and the gene variants 436 may be connected by edges to the transcripts 434 and peaks 438. It should be appreciated that the technology described herein may include graph structures describing relationships between four or more species, as embodiments of the technology are not limited in this respect.

As another example of a graph structure that may be generated using techniques described herein, FIG. 4C shows a single branch of a graph for one species in Cypher graph notation, in accordance with some embodiments described herein. The graph branch 400c may be, for example, describing only one of the first portion 410, second portion 420, or third portion 430 of FIG. 4A or 4B.

In some embodiments, the graph branch 400c shows graph objects notated within parentheses and edges notated within square brackets. The graph objects include genes, transcripts, variant effects, variants, and peaks. The edges between these graph objects may be described by relationships within the graph branch 400c. For example, the gene objects may be connected to variant objects by edges described by expression quantitative trait loci (eQTL) relationships. The gene objects may be connected to transcript objects by produces relationships (e.g., relationships indicating that a gene produces one or more particular transcripts). The transcript objects may be connected to variant objects by edges described by variant effect relationships (e.g., relationships indicating the effects a variant has on transcripts). The variant objects may be connected to peak objects by overlap relationships (e.g., relationships identifying DNA-binding sites determined using peak calling techniques). The gene objects of the graph branch 400c may be connected to another graph branch representing genomic relationships within a different species by edges described by homolog and orthologue relationships.

In some embodiments, the graph may be regenerated in response to new or updated genomic study data becoming available (e.g., as new experiments are performed and made public). Regenerating the graph may follow a same or similar process as initially generating the graph. For example, regenerating the graph may include accessing and obtaining the updated genomic study data from local and/or remote data sources. A subset of the updated genomic study data may be streamed onto local non-transitory computer-readable memory and/or RAM and stored as a cache. The stored subset may then be transformed into a database stored in one or more files having a uniform data format and being stored in non-transient computer-readable memory. Transforming the stored subset into the database may include determining graph objects and connections between graph objects. Once the database is finalized (e.g., after transforming one or more subsets of the genomic study data), the graph may be regenerated using the database.

Once the graph is generated or regenerated, a user may query the graph to identify, for one of the species in the graph, genes, gene variants, and/or associated information that is related to one or more gene or gene variants for another species in the graph. For example, a user may be studying certain gene variants associated with a disease in one species and may use the graph to identify genomic studies and/or treatment modalities tested on a model organism of another species and having gene variants related to the gene variants being studied by the user.

In some embodiments, a user may query the graph by “traversing” the graph from one species to another. For example, a user may provide a gene variant of a first species and, using gene-gene variant connections within the first species, may next identify the related gene of the first species. Then, the user may identify one or more related genes of the second species using the gene-gene connections that identify relationships between the first and second species. Finally, the user may identify one or more gene variants of the second species using the gene-gene variant connections of the second species. In this manner, the user may identify cross-species relations between gene variants and identify model organism studies that may be relevant to the user's needs.

The scale of the graphs 400a, 400b, and/or graph branch 400c can be determined by counting the number of distinct nodes and relationships for a species. Examples of scale for H. sapiens and M. musculus are provided in Table 1 herein.

TABLE 1

| | Node or | Measured Size | Approx. | Measured Size | Approx. |
|---|---|---|---|---|---|
| | Relationship | H. sapiens | Scale | M. musculus | Scale |
| h + | homolog and | 174,536 | 100k | 174,536 | 100k |
| r | orthologue | | | | |
| g | gene | 60,664 | 100k | 55,416 | 100k |
| e | eQTL | 70,910,867 | 100m | 68,593 | 100k |
| v | variant | 700,491,094 | 1b | 82,971,964 | 100m |
| p | produces | 236,920 | 100k | 142,434 | 100k |
| t | transcript | 236,920 | 100k | 142,434 | 100k |
| ve | variant effect | 4,099,787,379 | 1b | 293,827,773 | 100m |
| o | overlap | 29,198,149,596 | 10b | 1,301,223,594 | 1b |
| pk | peak | 169,479,076 | 100m | 140,392,506 | 100m |

As shown in Table 1, intersections of peaks by base pair with variants for H. sapiens are the largest relationship, with more than 29 billion entries. As additional experimental data continues to be collected, it should be anticipated that the scale of a graph will continue to increase.

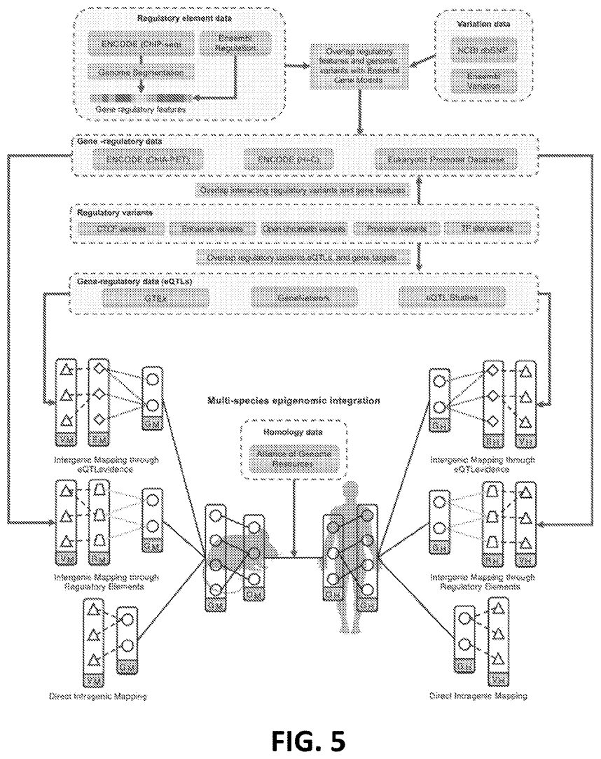

FIG. 5 shows an illustrative schematic diagram of a graph structure for a graph describing the relationships between gene variants of Mus musculus and Homo sapiens. In the diagram of FIG. 5, Human (V_{H}) and mouse (V_{M}) variants are connected to the respective gene (G_{M}, G_{H}) for each species. The genes (G_{M}, G_{H}) may either contain a coding variant or be regulated by a noncoding variant. Epigenetic markers and regulatory features (R_{M}, R_{H}) are retrieved from ENCODE and Ensembl, then overlapped with genetic variation data from Ensembl and the National Center for Biotechnology Information Single Nucleotide Polymorphism Database (NCBI dbSNP) in order to identify regulatory variants (V_{M}, V_{H}). The Regulatory variants (V_{M}, V_{H}) may be overlapped with gene-regulatory datasets in the form of cQTLs (E_{M}, E_{H}; processed from GTEx, GencNetwork, and specific mouse populations) and chromatin interaction studies (e.g., ChIA-PET experiments from ENCODE and genepromoter interactions from the Eukaryotic Promoter Database). Association of regulatory variants and gene-regulatory information allows for the identification of putative gene targets. These datasets are harmonized within-species for mice (V_{M}, E_{M}, G_{M}, R_{M}) and humans (V_{II}, E_{II}, G_{II}, R_{II}), then related across species through orthologous gene targets (O_{M}, O_{H}) derived from homology resources like the Alliance for Genome Resources.

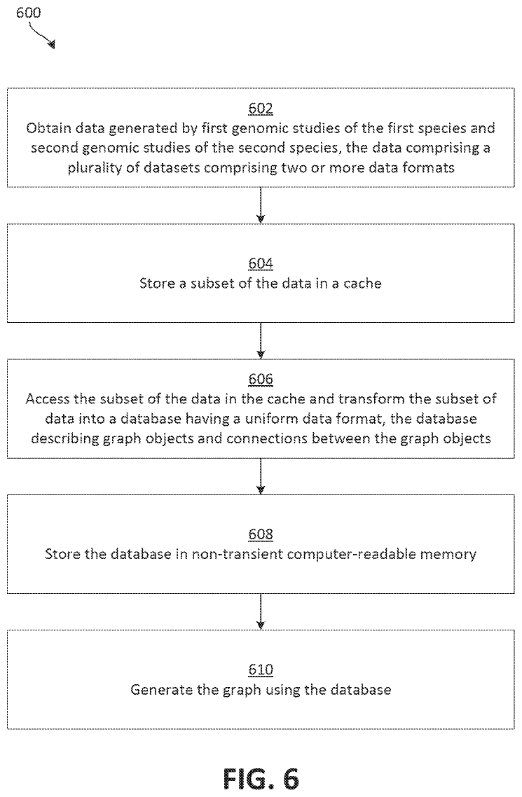

FIG. 6 is a flowchart of an illustrative process 600 for generating a graph, in accordance with some embodiments described herein. Process 600 may be implemented by a graph generation facility, such as the graph generation facility 112 of FIG. 1. As such, in some embodiments, the process 600 may be performed by a computing device configured to access and obtain genomic study data from locally-stored data (e.g., stored on a local computer memory) and/or from remotely-stored data (e.g., stored on remote computer memory, stored in the cloud, etc.). As another example, in some embodiments, the process 600 may be performed by one or more processors located remotely (e.g., as part of a cloud computing environment, as connected through a network) from the computing device accessed by an operator of the graph generation facility.

For ease of description, the process 600 will be described in connection with generating a graph describing relationships between gene variants of two species, but it should be appreciated the embodiments are not limited to generating graphs representing relationships between gene variants of two species and that some embodiments may generate graphs representing relationships between gene variants of three or more species.

In some embodiments, process 600 may begin at act 602, in which data generated by first genomic studies of a first species and by second genomic studies of a second species may be obtained. The data may be obtained by the computing device from remote and/or local data storage. For example, some portions of the data may be stored locally on the computer memory of the computing device and/or some portions of the data may be stored remotely (e.g., in a computer memory accessible over a network, in cloud computing storage, or otherwise available over the Internet).

In some embodiments, the data may comprise multiple datasets being stored using two or more data formats. For example, the genomic study data 202 may be stored in files stored using two or more of the following, non-limiting data formats: a gene transfer file (GTF) format, a genome variation format (GVF), a browser extensible data (BED) file format, an EXCEL binary file format (XLS), a comma-separate values (CSV) file format, a tab-separated values (TSV) file format, and/or a report (RPT) file format. It should be appreciated that the obtained data may be stored in files using alternative data formats than listed herein, as aspects of the technology described herein are not limited in this respect.

In some embodiments, the obtained data may include genomic study data related to two or more species of organisms. In some embodiments, the species may be selected from a list including the following species: Mus musculus, Homo sapiens, Rattus norvegicus, Danio rerio, Drosophilia melanogaster, Macaca mulatta, Caenorhabditis elegans, Saccharomyces cervisiae, Gallus gallus, and Canis familiaris. For example, in an embodiment where the obtained data may relate to two species, and the species may include Mus musculus and Homo sapiens. In some embodiments, the species may be selected from the species included in current and/or previous releases of Ensembl (https://www.ensembl.org/).

After obtaining the data, the process 600 may then move to act 604, in which a subset of the obtained data may be stored in a cache. The cache may be stored on any suitable combination of local non-transitory computer-readable memory (e.g., the local hard drive) and/or on local random-access memory (RAM). In some embodiments, storing the cache primarily or wholly on local computer-readable memory may enable generation of the graph using conventional computing resources (e.g., rather than requiring supercomputing resources).

After storing the subset of the obtained data in a cache, the process 600 may then move to act 606. In act 606, the subset of the data may be accessed from the cache and transformed into a database. The database may comprise one or more files having a uniform data format (e.g., CSV files). For example, the database may be stored in local non-transitory computer-readable memory (e.g., the local hard drive) and/or remote non-transitory computer-readable memory (e.g., on the cloud).

In some embodiments, transforming the subset of the obtained data into the database may include determining graph objects and connections between the graph objects to be included in the database. In some embodiments, determining the graph objects and/or connections may be performed using an object-relational mapping (ORM) tool. For example, a Java Persistence API (JPA) may be used in combination with an ORM tool (e.g., Hibernate) to transform the subset of the obtained data into the database including graph objects and/or connections.

In some embodiments, determining the graph objects may include determining a number of objects related to genes and gene variants. For example, the database may include gene objects, gene variant objects, and transcript objects as described herein. In some embodiments, determining the connections may include determining connections that describe relationships between different graph objects (e.g., gene-gene variant relationships, gene-gene relationships). For example, the database may include gene-gene variant relationships within each species and gene-gene relationships between species, as described herein.

The process 600 may then move to act 608, in which the database may be stored in non-transient computer-readable memory. For example, the database may be stored in local non-transient computer-readable memory (e.g., a local hard drive) and/or in remote non-transient computer-readable memory (e.g., on cloud storage).

In some embodiments, the database may be built in an iterative fashion by repeating acts 604, 606, and 608 until all of the obtained data has been incorporated into the database. For example, after transforming a first subset of the obtained data, the cached database storing the first subset may be deleted from local computer memory. Then, a second subset of the data may be streamed into a second cached database so that the second subset of the data may be transformed. The transformed second subset of data may be appended to the database. In this manner, the database may be built by streaming and transforming a number of subsets of the genomic study data.

After building the database, the process 600 may then move to act 610, in which the graph may be generated using the database. The graph may be generated as a weighted undirected graph, in some embodiments.

In some embodiments, the graph may be regenerated in response to new or updated genomic study data becoming available (e.g., as new experiments are performed and made public). Regenerating the graph may follow a same or similar process as initially generating the graph. For example, regenerating the graph may include accessing and obtaining the updated genomic study data from local and/or remote data sources. A subset of the updated genomic study data may be streamed onto local non-transitory computer-readable memory and/or RAM and stored as a cache. The stored subset may then be transformed into a database stored in one or more files having a uniform data format and being stored in non-transient computer-readable memory. Transforming the stored subset into the database may include determining graph objects and connections between graph objects. Once the database is finalized (e.g., after transforming one or more subsets of the genomic study data), the graph may be regenerated using the database.

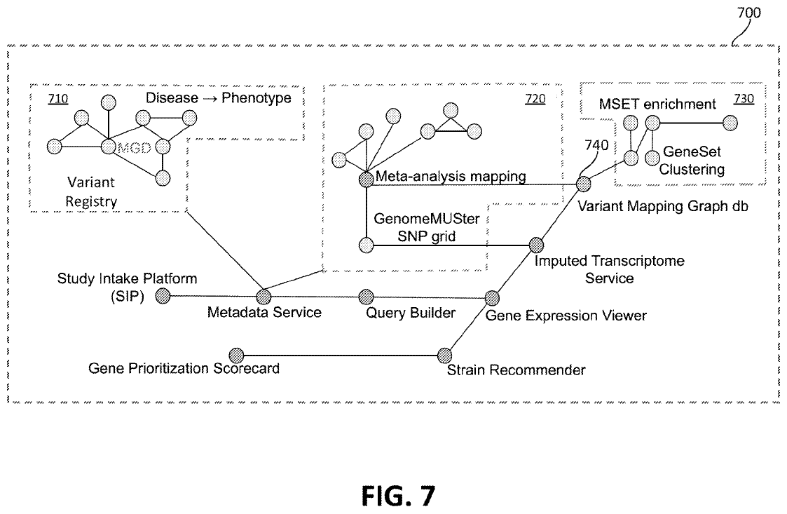

In some embodiments, a graph describing cross-species gene variant relationships may be integrated into a software suite for the study of complex disease in a target organism (e.g., Homo sapiens). The integrated software suite may be configured to provide cross-species links to enable researchers to identify relations between model organism characteristics, clinical data, experimental manipulations, genome sequences and features, complex genetic associations, and other biochemical data that may be relevant to the study of a target organism disease, disorder, or condition. An example of such an integrated software suite 700 is illustrated in FIG. 7.

In some embodiments, the integrated software suite 700 may include several modules 710, 720, and 730 linked by the gene variant graph 740. The modules may include a model organism gene variant registry 710, a model organism phenomics database 720, and a target organism functional genomics database 730. As one non-limiting example, the model organism gene variant registry 710 may be the Mouse Genome Informatics (MGI) database, the model organism phenomics database 720 may be the Mouse Phenome Database (MPD), and the target organism functional genomics database 730 may be Gene Weaver.

Including the gene variant graph 740 in the integrated software suite 700 may enable researchers to answer a variety of questions about gene variants in the target organism. For example, traversing the integrated software suite 700 to the model organism gene variant registry 710 may answer questions such as: (1) where is this gene expressed in the model organism? or (2) are there existing model organism models that could be used to design experiments to develop treatment modalities related to the target organism gene variant? As another example, traversing the integrated software suite 700 to the target organism functional genomics database 730 may answer questions such as: (1) what gene networks operate in a disease state? or (2) are there drugs or other treatments that target the gene product?

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be tangible (e.g., non-transitory) computer readable media. In some embodiments, the computer readable media may comprise a persistent memory.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Techniques operating according to the principles described herein may be implemented in any suitable manner. Included in the discussion above are a series of flow charts showing the steps and acts of various. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application, for example as a software program application such as a fetal cardiac analysis facility.

Some illustrative functional facilities have been described herein for carrying out one or more tasks. It should be appreciated, though, that the functional facilities and division of tasks described is merely illustrative of the type of functional facilities that may implement the techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

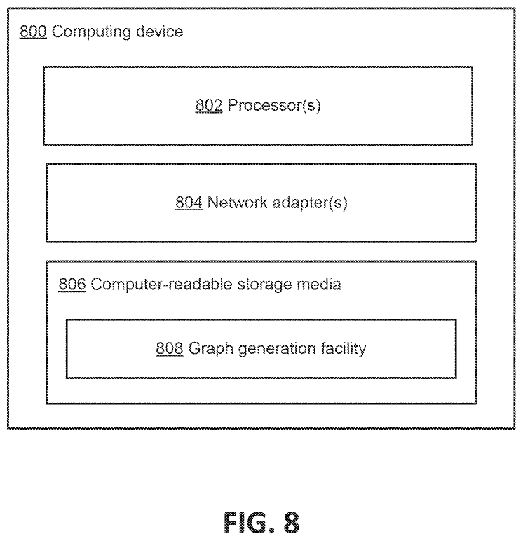

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 806 of FIG. 8 described below (i.e., as a portion of a computing device 800) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the illustrative computer system of FIG. 8, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing devices (co-located or geographically distributed) dedicated to executing the techniques described herein, one or more Field-Programmable Gate Arrays (FPGAs) for carrying out the techniques described herein, or any other suitable system.

FIG. 8 illustrates one exemplary implementation of a computing device in the form of a computing device 800 that may be used in a system implementing techniques described herein, although others are possible. It should be appreciated that FIG. 8 is intended neither to be a depiction of necessary components for a computing device to operate as a fetal cardiac MR analysis device and/or fetal cardiac MR image generator in accordance with the principles described herein, nor a comprehensive depiction.

Computing device 800 may comprise at least one processor 802, a network adapter 804, and computer-readable storage media 806. Computing device 800 may be, for example, a desktop or laptop personal computer, a personal digital assistant (PDA), a smart mobile phone, or any other suitable computing device. Network adapter 804 may be any suitable hardware and/or software to enable the computing device 800 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable storage media 806 may be adapted to store data to be processed and/or instructions to be executed by processor 802. Processor 802 enables processing of data and execution of instructions. The data and instructions may be stored on the computer-readable storage media 806.

The data and instructions stored on computer-readable storage media 806 may comprise computer-executable instructions implementing techniques which operate according to the principles described herein. In the example of FIG. 8, computer-readable storage media 806 stores computer-executable instructions implementing various facilities and storing various information as described above. Computer-readable storage media 806 may store graph generation facility 808 configured to generate graphs describing cross-species relationships between genes and/or gene variants.

While not illustrated in FIG. 8, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately” and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

What is claimed is:

1. A method of generating a graph comprising connections between a first set of gene variants associated with a first species and a second set of gene variants associated with a second species different than the first species, the method comprising:
using at least one computer hardware processor to perform:
obtaining data generated by first genomic studies of the first species and second genomic studies of the second species, the data comprising a plurality of datasets comprising two or more data formats;
storing a subset of the data in a cache;
accessing the subset of the data in the cache and transforming the subset of the data into a database having a uniform data format, the database describing graph objects and connections between the graph objects;
storing the database in non-transient computer-readable memory; and
generating the graph using the database.

2. The method of claim 1, wherein determining the graph objects and the connections comprises:
determining first gene objects and first gene variant objects associated with the first species;
determining second gene objects and second gene variant objects associated with the second species;
determining first connections between a gene object of the first gene objects and one or more gene variant objects of the first gene variant objects;
determining second connections between a gene object of the second gene objects and one or more gene variant objects of the second gene variant objects; and
determining third connections between the first gene objects and the second gene objects.

3. The method of claim 2, wherein determining the first and/or second connections comprises determining expression quantitative trait loci (eQTL), gene variant regulatory elements, chromatin contact regions, and/or intragenic mapping connections.

4. The method of claim 2 or 3, wherein determining the third connections comprises determining homolog and/or orthologue connections between the first and second species.

5. The method of any one of claims 1-4, wherein generating the graph comprises generating a weighted undirected graph.

6. The method of any one of claims 2-5, further comprising:
obtaining data generated by third genomic studies of a third species, the third species being different than the first species or the second species;
determining third gene objects and third gene variant objects associated with the third species;
determining fourth connections between a gene object of the third gene objects and one or more gene variant objects of the third gene variant objects; and
determining fifth connections between the third gene objects and the first or second gene objects.

7. The method of any one of claims 1-6, further comprising identifying, using the graph and a gene variant object associated with the first species, one or more genomic studies associated with the second species.

8. The method of any one of claims 1-7, wherein using the graph to identify the one or more genomic studies comprises:
identifying, using the gene variant object associated with the first species, a gene object of the first gene objects using the first connections;
identifying a gene object of the second gene objects using the third connections;
identifying a gene variant object associated with the second species using the second connections; and
identifying the one or more genomic studies using the gene variant associated with the identified gene variant object.

9. The method of any one of claims 1-8, wherein the gene variant object associated with the first species is associated with a disease, and wherein the method further comprises identifying a treatment modality for a patient having the disease using the identified one or more genomic studies.

10. The method of any one of claims 1-9, wherein the first species and the second species comprise two species selected from a list including: Mus musculus, Homo sapiens, Rattus norvegicus, Danio rerio, Drosophilia melanogaster, Macaca mulatta, Caenorhabditis elegans, Saccharomyces cervisiae, Gallus gallus, and Canis familiaris.

11. The method of any one of claims 1-10, wherein the first species and the second species comprise Mus musculus and Homo sapiens.

12. The method of any one of claims 1-11 or any other preceding claim, wherein transforming the subset of data into a uniform data format comprises transforming the subset of data into one or more comma-separated values (CSV) files.

13. The method of any one of claims 1-12, wherein obtaining the data comprising two or more data formats comprises obtaining data comprising two or more of a gene transfer file (GTF) format, a genome variation format (GVF), browser extensible data (BED) file format, an EXCEL binary file format (XLS), a comma-separate values (CSV) file format, a tab-separated values (TSV) file format, and/or a report (RPT) file format.

14. The method of any one of claims 1-13, further comprising regenerating the graph based on updated data, the regenerating comprising:
obtaining the updated data generated by first genomic studies of the first species and second genomic studies of the second species, the updated data comprising a plurality of datasets comprising two or more data formats;
storing a subset of the updated data in a cache;
accessing the subset of the updated data in the cache and transforming the subset of the updated data into a uniform data format, the uniform data format describing graph objects and connections between the graph objects;
storing, in non-transient computer-readable memory, the transformed subset of the updated data in a database; and
regenerating the graph using the database.

15. The method of any one of claims 1-14, wherein determining the graph objects and the connections comprises:
determining first transcript objects associated with the first species;
determining sixth connections between the first transcript objects and the first gene objects; and
determining seventh connections between the first transcript objects and the first gene variant objects.

16. The method of any one of claims 1-15, wherein determining the graph objects and the connections comprises:
determining first peak objects associated with the first species; and
determining eighth connections between the first peak objects and the first gene variant objects.

17. At least one non-transitory computer readable storage medium storing processor-executable instructions that, when executed by at least one processor, cause the at least one processor to perform a method of generating a graph comprising connections between a first set of gene variants associated with a first species and a second set of gene variants associated with a second species, the method comprising:
obtaining data generated by first genomic studies of the first species and second genomic studies of the second species, the data comprising a plurality of datasets comprising two or more data formats;
storing a subset of the data in a cache;
accessing the subset of the data in the cache and transforming the subset of the data into a database having a uniform data format, the uniform data format describing graph objects and connections between the graph objects;
storing the database in non-transient computer-readable memory; and
generating the graph using the database.

18. The at least one non-transitory computer readable storage medium of claim 17, wherein determining the graph objects and the connections comprises:
determining first gene objects and first gene variant objects associated with the first species;
determining second gene objects and second gene variant objects associated with the second species;
determining first connections between a gene object of the first gene objects and one or more gene variant objects of the first gene variant objects;
determining second connections between a gene object of the second gene objects and one or more gene variant objects of the second gene variant objects; and
determining third connections between the first gene objects and the second gene objects.

19. The at least one non-transitory computer readable storage medium of claim 18, wherein determining the first and/or second connections comprises determining expression quantitative trait loci (eQTL), gene variant regulatory elements, chromatin contact regions, and/or intragenic mapping connections.

20. The at least one non-transitory computer readable storage medium of claim 18 or 19, wherein determining the third connections comprises determining homolog and/or orthologue connections between the first and second species.

21. The at least one non-transitory computer readable storage medium of any one of claims 17-20, wherein generating the graph comprises generating a weighted undirected graph.

22. The at least one non-transitory computer readable storage medium of any one of claims 19-21, further comprising:
obtaining data generated by third genomic studies of a third species, the third species being different than the first species or the second species;
determining third gene objects and third gene variant objects associated with the third species;
determining fourth connections between a gene object of the third gene objects and one or more gene variant objects of the third gene variant objects; and
determining fifth connections between the third gene objects and the first or second gene objects.

23. The at least one non-transitory computer readable storage medium of any one of claims 17-22, further comprising identifying, using the graph and a gene variant object associated with the first species, one or more genomic studies associated with the second species.

24. The at least one non-transitory computer readable storage medium of any one of claims 17-23, wherein using the graph to identify the one or more genomic studies comprises:
identifying, using the gene variant object associated with the first species, a gene object of the first gene objects using the first connections;
identifying a gene object of the second gene objects using the third connections;
identifying a gene variant object associated with the second species using the second connections; and
identifying the one or more genomic studies using the gene variant associated with the identified gene variant object.

25. The at least one non-transitory computer readable storage medium of any one of claims 17-24, wherein the gene variant object associated with the first species is associated with a disease, and wherein the method further comprises identifying a treatment modality for a patient having the disease using the identified one or more genomic studies.

26. The at least one non-transitory computer readable storage medium of any one of claims 17-25, wherein the first species and the second species comprise two species selected from a list including: Mus musculus, Homo sapiens, Rattus norvegicus, Danio rerio, Drosophilia melanogaster, Macaca mulatta, Caenorhabditis elegans, Saccharomyces cervisiae, Gallus gallus, and Canis familiaris.

27. The at least one non-transitory computer readable storage medium of any one of claims 17-26, wherein the first species and the second species comprise Mus musculus and Homo sapiens.

28. The at least one non-transitory computer readable storage medium of any one of claims 17-27 or any other preceding claim, wherein transforming the subset of data into a uniform data format comprises transforming the subset of data into one or more comma-separated values (CSV) files.

29. The at least one non-transitory computer readable storage medium of any one of claims 17-28, wherein obtaining the data comprising two or more data formats comprises obtaining data comprising two or more of a gene transfer file (GTF) format, a genome variation format (GVF), browser extensible data (BED) file format, an EXCEL binary file format (XLS), a comma-separate values (CSV) file format, a tab-separated values (TSV) file format, and/or a report (RPT) file format.

30. The at least one non-transitory computer readable storage medium of any one of claims 17-29, the method further comprising regenerating the graph based on updated data, the regenerating comprising:
obtaining the updated data generated by first genomic studies of the first species and second genomic studies of the second species, the updated data comprising a plurality of datasets comprising two or more data formats;
storing a subset of the updated data in a cache;
accessing the subset of the updated data in the cache and transforming the subset of the updated data into a uniform data format, the uniform data format describing graph objects and connections between the graph objects;
storing, in non-transient computer-readable memory, the transformed subset of the updated data in a database; and
regenerating the graph using the database.

31. The at least one non-transitory computer readable storage medium of any one of claims 17-30, wherein determining the graph objects and the connections comprises:
determining first transcript objects associated with the first species;
determining sixth connections between the first transcript objects and the first gene objects; and
determining seventh connections between the first transcript objects and the first gene variant objects.

32. The at least one non-transitory computer readable storage medium of any one of claims 17-31, wherein determining the graph objects and the connections comprises:
determining first peak objects associated with the first species; and
determining eighth connections between the first peak objects and the first gene variant objects.

33. A system for generating a graph comprising connections between a first set of gene variants associated with a first species and a second set of gene variants associated with a second species, the system comprising:
at least one processor; and
at least one non-transitory computer readable storage medium storing processor-executable instructions that, when executed by the at least one processor, cause the at least one processor to perform a method comprising:
obtaining data generated by first genomic studies of the first species and second genomic studies of the second species, the data comprising a plurality of datasets comprising two or more data formats;
storing a subset of the data in a cache;
accessing the subset of the data in the cache and transforming the subset of the data into a database having a uniform data format, the uniform data format describing graph objects and connections between the graph objects;
storing the database in non-transient computer-readable memory; and
generating the graph using the database.

34. The system of claim 33, wherein determining the graph objects and the connections comprises:
determining first gene objects and first gene variant objects associated with the first species;
determining second gene objects and second gene variant objects associated with the second species;
determining first connections between a gene object of the first gene objects and one or more gene variant objects of the first gene variant objects;
determining second connections between a gene object of the second gene objects and one or more gene variant objects of the second gene variant objects; and
determining third connections between the first gene objects and the second gene objects.

35. The system of claim 33 or 34, wherein determining the first and/or second connections comprises determining expression quantitative trait loci (eQTL), gene variant regulatory elements, chromatin contact regions, and/or intragenic mapping connections.

36. The system of any one of claim 34 or 35, wherein determining the third connections comprises determining homolog and/or orthologue connections between the first and second species.

37. The system of any one of claims 33-36, wherein generating the graph comprises generating a weighted undirected graph.

38. The system of any one of claims 35-37, the method further comprising:
obtaining data generated by third genomic studies of a third species, the third species being different than the first species or the second species;
determining third gene objects and third gene variant objects associated with the third species;
determining fourth connections between a gene object of the third gene objects and one or more gene variant objects of the third gene variant objects; and
determining fifth connections between the third gene objects and the first or second gene objects.

39. The system of any one of claims 33-38, the method further comprising identifying, using the graph and a gene variant object associated with the first species, one or more genomic studies associated with the second species.

40. The system of any one of claims 33-39, wherein using the graph to identify the one or more genomic studies comprises:
identifying, using the gene variant object associated with the first species, a gene object of the first gene objects using the first connections;
identifying a gene object of the second gene objects using the third connections;
identifying a gene variant object associated with the second species using the second connections; and
identifying the one or more genomic studies using the gene variant associated with the identified gene variant object.

41. The system of any one of claims 33-40, wherein the gene variant object associated with the first species is associated with a disease, and wherein the method further comprises identifying a treatment modality for a patient having the discasc using the identified one or more genomic studies.

42. The system of any one of claims 33-41, wherein the first species and the second species comprise two species selected from a list including: Mus musculus, Homo sapiens, Rattus norvegicus, Danio rerio, Drosophilia melanogaster, Macaca mulatta, Caenorhabditis elegans, Saccharomyces cervisiae, Gallus gallus, and Canis familiaris.

43. The system of any one of claims 33-42, wherein the first species and the second species comprise Mus musculus and Homo sapiens.

44. The system of any one of claims 33-43 or any other preceding claim, wherein transforming the subset of data into a uniform data format comprises transforming the subset of data into one or more comma-separated values (CSV) files.

45. The system of any one of claims 33-44, wherein obtaining the data comprising two or more data formats comprises obtaining data comprising two or more of a gene transfer file (GTF) format, a genome variation format (GVF), browser extensible data (BED) file format, an EXCEL binary file format (XLS), a comma-separate values (CSV) file format, a tab-separated values (TSV) file format, and/or a report (RPT) file format.

46. The system of any one of claims 33-45, the method further comprising regenerating the graph based on updated data, the regenerating comprising:
obtaining the updated data generated by first genomic studies of the first species and second genomic studies of the second species, the updated data comprising a plurality of datasets comprising two or more data formats;
storing a subset of the updated data in a cache;
accessing the subset of the updated data in the cache and transforming the subset of the updated data into a uniform data format, the uniform data format describing graph objects and connections between the graph objects;
storing, in non-transient computer-readable memory, the transformed subset of the updated data in a database; and
regenerating the graph using the database.

47. The system of any one of claims 33-46, wherein determining the graph objects and the connections comprises:
determining first transcript objects associated with the first species;
determining sixth connections between the first transcript objects and the first gene objects; and
determining seventh connections between the first transcript objects and the first gene variant objects.

48. The system of any one of claims 33-47, wherein determining the graph objects and the connections comprises:
determining first peak objects associated with the first species; and
determining eighth connections between the first peak objects and the first gene variant objects.
